(19) United States
(12) Patent Application Publication
(10) Pub. No.: US 2023/0390504 A1
(43) Pub. Date: Dec. 7, 2023

(54) Battery-Free Sensing Solution for Drug Delivery Devices

(71) Applicant: Becton, Dickinson and Company, Franklin Lakes, NJ (US)

(72) Inventor: Olivier Albenge, Echirolles (FR)

(21) Appl. No.: 18/203,951

(22) Filed: May 31, 2023

(30) Foreign Application Priority Data
Jun. 1, 2022 (EP) 22176651.2

(51) Int. Cl. A61M 5/32; A61M 5/31

(52) U.S. Cl. CPC A61M 5/3287; A61M 5/3129

(57) ABSTRACT

Provided herein is a drug delivery device including a syringe barrel having a proximal end, a distal end, and a sidewall arranged between the proximal end and the distal end defining an interior configured to hold a composition, a plunger rod received at least partially within the interior of the syringe, the plunger rod having a proximal end, a distal end, and a stopper arranged at the distal end, and a sensor assembly, including a capacitive element and a short-range communication device in electrical communication with the capacitive element, wherein the short-range communication device is capable of measuring capacitance at the capacitive element.

### BACKGROUND OF THE INVENTION

### Field of the Invention

The present invention relates generally to drug delivery devices and components therefor, and in particular, to components allowing for detection and communication of the injection status of drug delivery devices.

### Description of Related Art

Drug delivery devices have traditionally lacked features that would allow a healthcare provider to confirm appropriate administration of medicaments. Ensuring timely, appropriate administration of some medicaments can be critical, particularly, for example, in the instance of the evaluation of new drugs in clinical trials, where accurate information is essential.

While some devices provide the ability to communicate certain types of information, for example as described in International Patent Application Publication Nos. WO 2016/087512, WO 2017/070391, WO 2018/111969, WO 2018/213837, and WO 2021/094797, and in U.S. Patent Application Publication Nos. 2019/0083708, 2019/0321555, and 2019/0344019, a need exists in the art for a cost-effective device that accurately detects injection status, and can communicate this drug delivery information to appropriate stakeholders.

### SUMMARY OF THE INVENTION

Provided herein is a drug delivery device including a syringe barrel having a proximal end, a distal end, and a sidewall arranged between the proximal end and the distal end defining an interior configured to hold a composition, a plunger rod received at least partially within the interior of the syringe, the plunger rod having a proximal end, a distal end, and a stopper arranged at the distal end, and a sensor assembly, including a capacitive element and a short-range communication device in electrical communication with the capacitive element, wherein the short-range communication device is capable of measuring capacitance at the capacitive element.

Also provided herein is a system including a drug delivery device including a syringe barrel having a proximal end, a distal end, and a sidewall arranged between the proximal end and the distal end defining an interior configured to hold a composition, a plunger rod received at least partially within the interior of the syringe, the plunger rod having a proximal end, a distal end, and a stopper arranged at the distal end, and a sensor assembly, including a capacitive element and a short-range communication device in electrical communication with the capacitive element, wherein the short-range communication device is capable of measuring capacitance at the capacitive element. The system also includes an NFC-enabled mobile device.

Also provided herein is a method of monitoring drug delivery status, including steps of providing a drug delivery device including a syringe barrel having a proximal end, a distal end, a needle arranged at the distal end, and a sidewall arranged between the proximal end and the distal end defining an interior configured to hold a composition, a plunger rod received at least partially within the interior of the syringe, the plunger rod having a proximal end, a distal end, and a stopper arranged at the distal end, and a sensor assembly, including a capacitive element and a short-range communication device in electrical communication with the capacitive element, wherein the short-range communication device is capable of measuring capacitance at the capacitive element, powering, with a mobile device, the sensor assembly to obtain first capacitance data, and transmitting, with the short-range communication device and to the mobile device, the first capacitance data.

### BRIEF DESCRIPTION OF THE DRAWINGS

FIGS. 1A and 1B are perspective and cross-sectional views of a drug delivery device according to non-limiting embodiments described herein;

FIGS. 2A and 2B are perspective and cross-sectional views of a drug delivery device according to non-limiting embodiments described herein;

FIGS. 3A and 3B are side views of a drug delivery device according to non-limiting embodiments described herein;

FIG. 4 is a schematic diagram of a system according to non-limiting embodiments described herein;

FIG. 5 is a diagram of a non-limiting embodiment of an environment in which systems, devices, and/or methods described herein may be implemented; and

FIG. 6 is a diagram of a non-limiting embodiment of components of one or more devices of FIG. 5.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible. Any known electronic communication protocols, including proprietary communication protocols, and/or algorithms can be used such as, for example, TCP/IP (including HTTP and other protocols), WLAN (including 502.11a/b/g/n and other radio frequency-based protocols and methods), analog transmissions, Global System for Mobile Communications (GSM), UltraWideB and (UWB), 3G/4G/LTE, BLUETOOTH, ZigBee, EnOcean, TransferJet, Wireless USB, and the like known to those of skill in the art. In some non-limiting embodiments, a message may refer to a network packet (e.g., a data packet and/or the like) that includes data.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, “electrical communication,” refers to a state of communication, for example through a conductive lead, antenna, etc., between one or more capacitive elements, such as a capacitor, and a short-range communication device, such as an NFC or RFID chip/tag, whereby the NFC or RFID chip/tag is capable of detecting and/or measuring a change in capacitance at the capacitive element(s).

Provided herein are drug delivery devices, systems including drug delivery devices, and methods using such systems and devices. Drug delivery devices described herein, as well as systems and methods including the same, include a sensor assembly including a short-range communication device to allow for monitoring of status of the drug delivery device, and communication of that status.

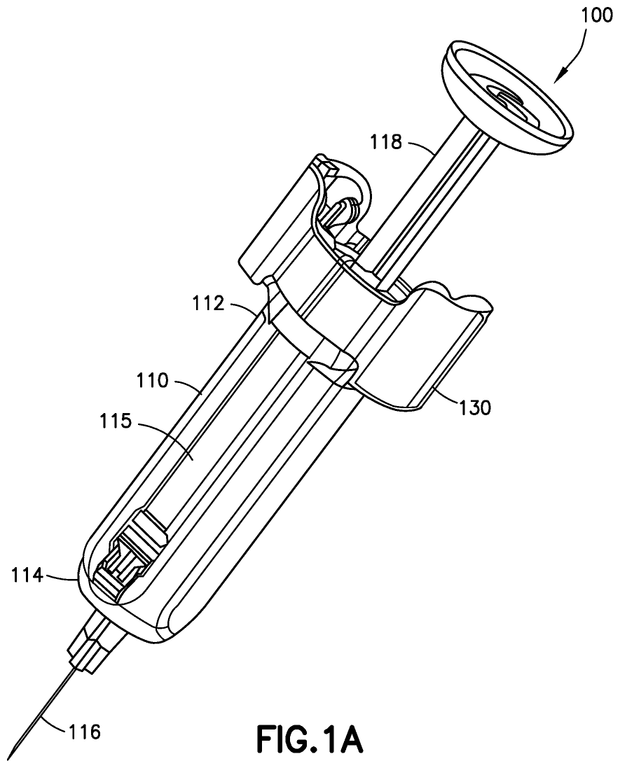

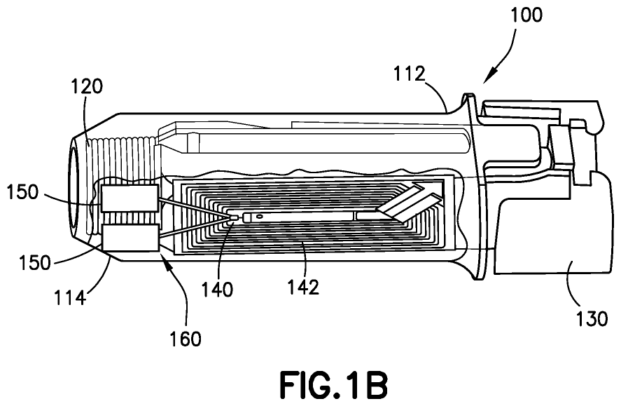

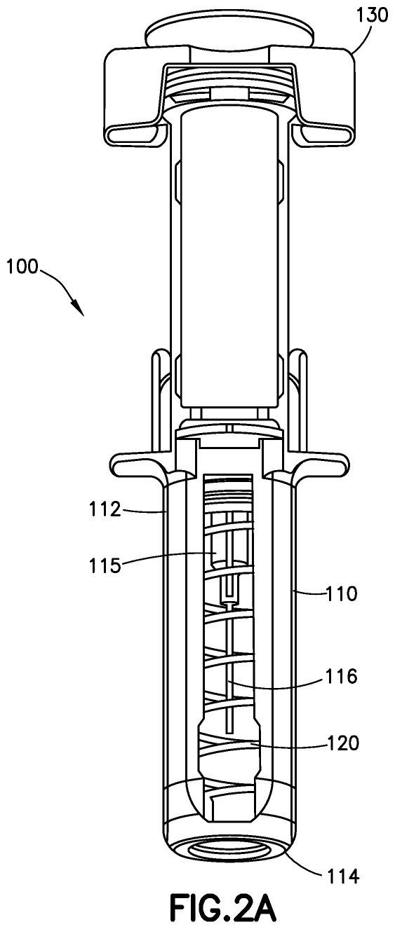

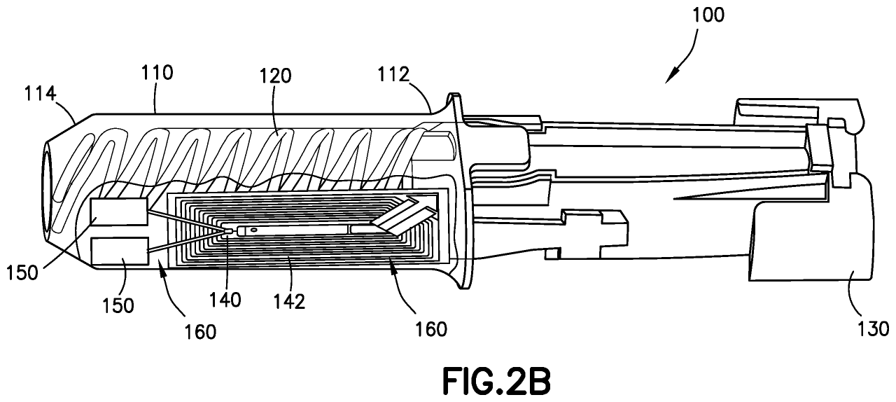

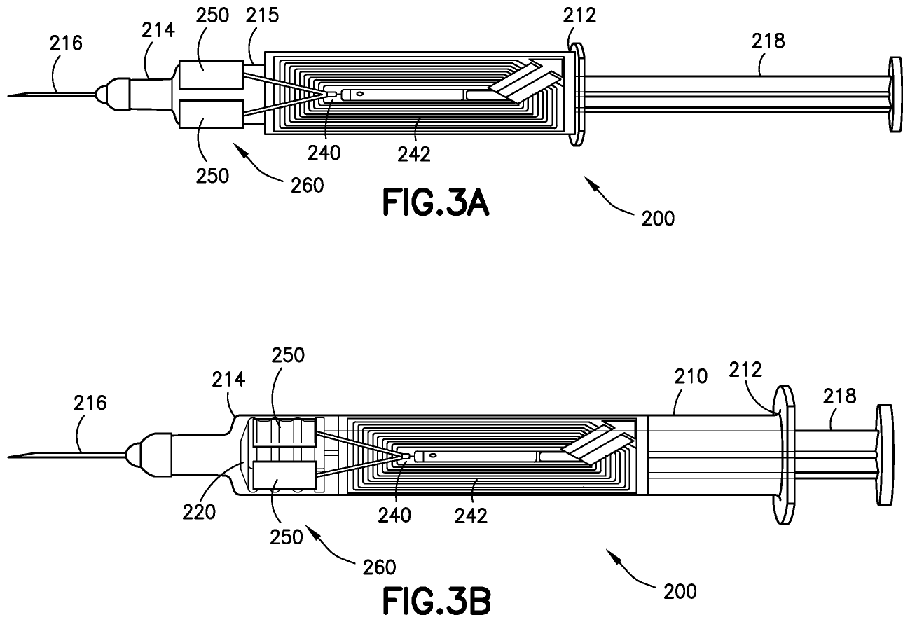

Turning to FIGS. 1A-2B, shown are non-limiting embodiments of a drug delivery device 100. Drug delivery device 100, while shown as a device similar to that disclosed in U.S. Pat. No. 10,702,663, which is incorporated by reference herein in its entirety, may be any drug delivery device, such as a manual syringe (as shown in FIGS. 3A and 3B), an autoinjector, a pen injector, or a wearable pump (e.g., an on-body injector). In the non-limiting embodiment shown in FIGS. 1A-2B, drug delivery device 100 includes a syringe 115 having a proximal end, a distal end, a sidewall therebetween defining an interior configured to hold a composition (e.g., a therapeutic composition) and a needle 116 arranged at the distal end in fluid communication with the interior. Drug delivery device 100 may also include a plunger rod 118 having a proximal end (optionally including a thumb pad), a distal end, and a stopper arranged at distal end thereof. Drug delivery device 100 may include a displaceable needle shield 110 having a proximal end 112, a distal end 114, and a spring 120 configured to displace needle shield 110 from a first, proximal position in which needle shield 110 does not surround needle 116 to a second, distal position in which needle shield 110 surrounds needle 116. In the non-limiting embodiment of FIGS. 1A-2B, drug delivery device 100 further includes a clip 130, which may be configured to prevent removal of plunger rod 118 from drug delivery device 100.

With reference to FIGS. 1B and 2B, drug delivery device 100 is shown in a pre-use (FIG. 1B) and post-use (FIG. 2B) configuration, together with a short-range communication device 140 and one or more capacitive elements 150 (not visible in FIGS. 1A and 2A). Short-range communication device 140 and one or more capacitive elements 150 may be considered to be a sensor assembly 160, and may be provided on an internal surface of needle shield 110 or an outer surface of syringe 115, for example as a label. While the accompanying Figures illustrate an exemplary embodiment of sensor assembly 160, those of skill will appreciate that sensor assembly 160 may be arranged on an outer surface of needle shield 110, or embedded within needle shield 110, for example through an in-mold labelling process. This arrangement of sensor assembly 160 allows for determination of the use status of drug delivery device 100 without the need for the sensor assembly 160 to physically contact any other component of the drug delivery device 100 (e.g., no portion of the sensor assembly 160 need be acted upon physically). Sensor assembly 160 may further include one or more processors in communication with short-range communication device 140 and/or memory. In non-limiting embodiments, short-range communication device 140 is a near-field communication (NFC) device. Suitable NFC devices and components therefor are known in the art and are commercially available, for example from NXP Semiconductors (Eindhoven, The Netherlands) and Identiv (Santa Anna, CA). Suitable short-range communication devices may include a chip/tag (optionally including a processor, memory, a cryptographic security processor, and/or one or more additional sensors, such as a temperature sensor), for example for storage of data and/or instructions, and an antenna for communication with NFC-enabled devices, such as computing devices described herein. In non-limiting embodiments, sensor assembly 160 is unpowered (e.g., does not include an internal power source, such as a battery). In non-limiting embodiments, sensor assembly 160 is powered by an external source, for example an NFC-enabled computing device as described herein. As NFC technology allows for powering of the short-range communication device wirelessly, in non-limiting embodiments, data is collected and transmitted without the need to form/break any electrical circuits, and without any contact between drug delivery device 100 and any external device, such as a user's computing device (e.g., a mobile device). The present disclosure also contemplates embodiments where an NFC device is provided inside of a drug delivery device 100, for example, a reusable drug delivery device 100 is contemplated, where an NFC-containing component may be included on or within housing, and a syringe can be inserted/removed from the housing. In non-limiting embodiments, sensor assembly further includes a second communication device, such as an RFID device. In non-limiting embodiments, sensor assembly 160 includes, as short range communication device 140, a dual-frequency NFC chip, which includes multiple antennae, including at least one for NFC and one for RFID communication.

As noted above, sensor assembly 160 includes at least one capacitive element 150. In non-limiting embodiments, at least one capacitive element 150 includes a pair of capacitor plates. Suitable materials for capacitor plates are known to those of skill in the art. Those of skill in the art will also appreciate that the electrical field stored by a capacitor is influenced by the arrangement of the capacitor (e.g., size of and distance between capacitor plates) and the local environment, including materials surrounding and/or adjacent to the capacitor. As the capacitive element 150 is in electrical communication (e.g., via one or more leads) with short-range communication device 140 (and any optional further communication devices, such as an RFID device), short-range communication device (e.g., NFC chip) 140 can, when powered, detect and/or determine capacitance at the capacitive element 150, which, as noted, may be affected by surrounding/adjacent materials. In non-limiting embodiments, sensor assembly 160 is arranged at or near distal end 114 of needle shield 110. In non-limiting embodiments, capacitive element 150 includes a pair of capacitor plates arranged parallel to one another. In non-limiting embodiments, capacitive element 150 includes capacitor plates separated from one another by air or an insulating material, such as ceramic, glass, paper, and/or plastic.

For example, with reference to FIGS. 1A-2B, capacitive element 150 is, in a pre-use position, arranged adjacent to the material (e.g., plastic) of needle shield 110, syringe 115 barrel (e.g., plastic or glass), and any composition received within the interior of syringe 115, as well as spring 120, which in a pre-use position of drug delivery device 100 is compressed (e.g., there is a larger density of spring material (e.g., metal) in the pre-use position than in the post-use position). Further, in the post-use position, capacitive element 150 is adjacent to the material (e.g., plastic) of needle shield 110, syringe 115 barrel (e.g., plastic or glass), (rather than any composition received within the interior of syringe 115), as well as the lower density of spring material as spring 120 is in an expanded configuration. Such a change in environment affects the capacitance of capacitive element 150, which may be detected when power is supplied to sensor assembly, for example by an NFC-enabled computing device associated with a user of drug delivery device 100. Such capacitance data may then, when power is supplied to sensor assembly 160, be stored by short-range communication device 140, and/or communicated, for example through NFC and/or RFID, to a computing device computing device associated with a user of drug delivery device 100. A user may, with an NFC-enabled computing device, provide power to the sensor assembly 160 and obtain capacitance data before and/or after drug delivery.

Turning to FIGS. 3A and 3B, shown is a non-limiting embodiment of a drug delivery device 200 with a short-range communication device 240 and capacitive element 250 (e.g., sensor assembly 260). Drug delivery device 200 in the illustrated embodiment of FIGS. 3A and 3B is a manual syringe, but those of skill will appreciate that the concepts described herein may be applicable to any drug container, such as a pre-filled syringe, for example for use in a pen or autoinjector, pump device, on-body injector, or other drug delivery device. Drug delivery device 200 in the embodiment of FIGS. 3A and 3B includes a syringe barrel 210 having a proximal end 212, a distal end 214, and a sidewall therebetween defining an interior configured to hold a composition. Drug delivery device 200 may include at distal end 214 thereof a needle 216 in fluid communication with interior, and, at proximal end 212, a plunger rod 218 that may include a thumb pad at a proximal end thereof and a stopper 220 at distal end thereof.

As shown in FIGS. 3A and 3B, sensor assembly 260 may be arranged at or near distal end 214 of drug delivery device 200, such that, for example as described above, capacitive element 250 is, in a pre-use position, arranged adjacent to syringe barrel 210 (e.g., plastic or glass), and any composition received within the interior of syringe barrel 210. In the post-use position, capacitive element 250 is adjacent to syringe barrel 210 (e.g., plastic or glass) and the material of the stopper (rather than any composition received within the interior of syringe barrel 210). Such a change in environment affects the capacitance of capacitive element 250, which may be detected when power is supplied to sensor assembly 260, for example by an NFC-enabled computing device associated with a user of drug delivery device 200. Such capacitance data may then, when power is supplied to sensor assembly 260, be stored by short-range communication device 240, and/or communicated, for example through NFC and/or RFID, to a computing device computing device associated with a user of drug delivery device 200. A user may, with an NFC-enabled computing device, provide power to the sensor assembly 260 and obtain capacitance data before and/or after drug delivery.

While the illustrated embodiments (e.g., FIGS. 1A-3B) show an arrangement where sensor assembly 160/260 is arranged such that capacitive element 150/250 is located at or near a distal end 114/214 of drug delivery device 100/200, those of skill will appreciate that sensor assembly 160/260 may alternatively, or in addition, be arranged such that capacitive element 150/250 is located at or near proximal end 112/212 of drug delivery device 100/200. Those of skill will appreciate that the difference in capacitance described above as providing indication of the status of drug delivery device 100/200 will be applicable regardless of the arrangement of capacitive element 150/250.

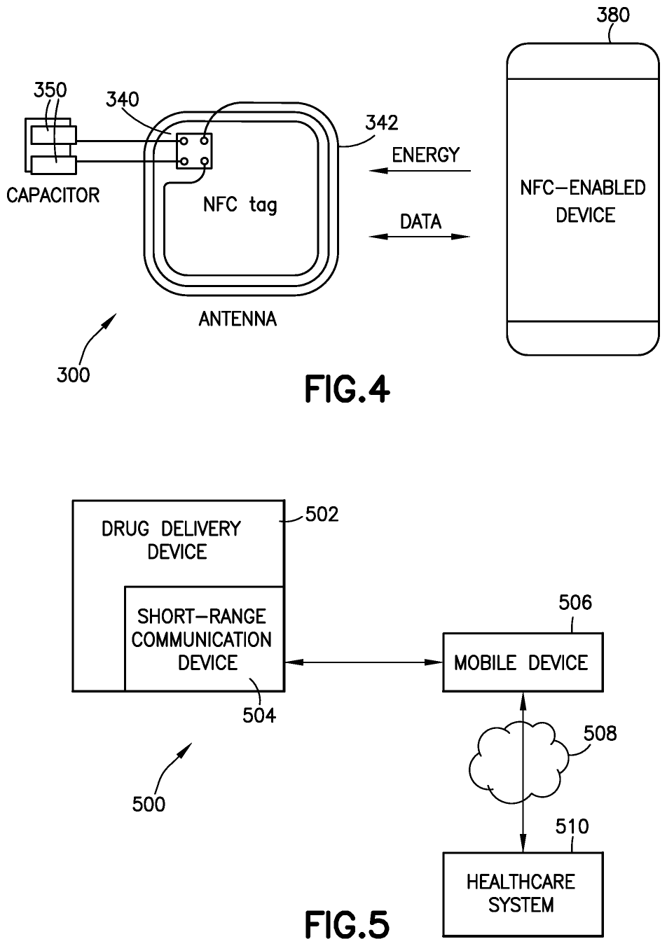

Turning to FIG. 4, shown is an example system 300 environment, including short-range communication device 340, with antenna 342 fully visible due to the close-up perspective, capacitive element 350, and computing device 380 (in the form of a mobile device). Those of skill will appreciate that while not described in detail above, short-range communication devices useful with the aforementioned embodiments may include an antenna. Computing device 380 may be NFC (and, optionally, RFID)-enabled, such that, by bringing computing device 380 close to short-range communication device 340, energy is provided to short-range communication device 340, and capacitance at capacitive element 350 can be detected and/or determined. As capacitance is detected and/or determined, capacitance data detected and/or generated by short-range communication device 340 may be transmitted from short-range communication device 340 to computing device 380.

Referring now to FIG. 5, shown is a diagram of an example environment 500 in which devices, systems, and/or methods, described herein, may be implemented. As shown in FIG. 5, the environment 500 can include drug delivery device 502 including short-range communication device 504, user device 506 (e.g., a user's mobile device), healthcare system 510, and/or communication network 508. Drug delivery device 502, user device 506, and healthcare system 510 may interconnect (e.g., establish a connection to communicate) via wired connections, wireless connections, or a combination of wired and wireless connections.

Drug delivery device 502, which can be a rigid drug container, a syringe, autoinjector, wearable injector, and/or pump as described herein, can include, as described above, a sensor assembly including short-range communication device 504 and capacitive element(s). As described above, drug delivery device 502 can be configured to communicate with a user device 506.

User device 506 can be a computing device as described herein, in some non-limiting embodiments, a smartphone. User device 506 can be programmed or configured to communicate, for example through communication network 508, with a healthcare system 510, for example through a mobile application executable on user device 506.

Communication network 508 may include one or more wired and/or wireless networks. For example, communication network 508 may include a BLUETOOTH connection (e.g., between drug delivery device 502 and user device 506), a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a UWB network, a local area network (LAN), a low-power wide area network (LPWAN), a wide area network (WAN), a metropolitan area network (MAN), a telephone network (e.g., the public switched telephone network (PSTN) and/or the like), a private network, an ad hoc network, an intranet, the Internet, a fiber optic-based network, a cloud computing network, and/or the like, and/or a combination of some or all of these or other types of networks.

Healthcare system 510 may include a server, a group of servers, and/or other like devices. More than one healthcare system 510 can be provided, for example, a system associated with a device manufacturer, a system associated with a pharmaceutical manufacturer, a system associated with a healthcare provider, a system associated with a government agency, and/or a system associated with a study sponsor, for example a sponsor of a clinical trial.

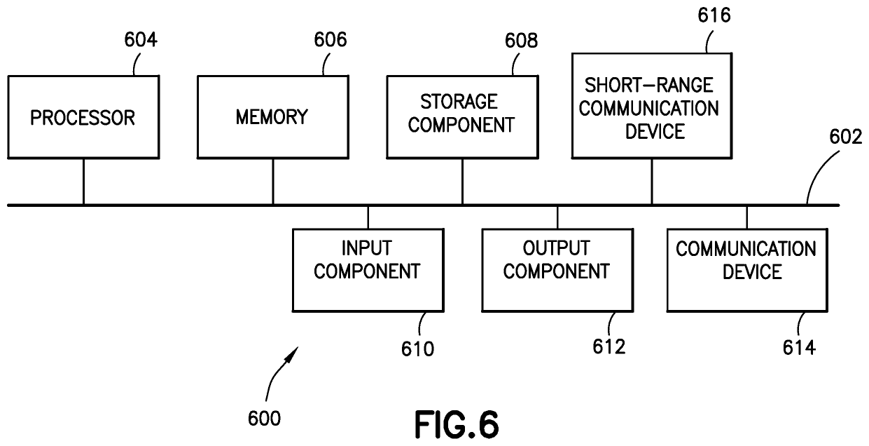

Referring now to FIG. 6, shown is a diagram of example components of an exemplary computing device 600, in an exemplary system, useful for methods described herein. Such a computing device 600 may correspond to a component within the drug delivery device as described herein, a user device as described herein, and/or a healthcare system as described herein. As shown in FIG. 6, a computing device 600 may include bus 602, processor 604, memory 606, storage component 608, input component 610, output component 612, and/or communication interface 614.

Bus 602 may include a component that permits communication among the components of a computing device 600. In some non-limiting embodiments, processor 604 may be implemented in hardware, software, or a combination of hardware and software. For example, processor 604 may include a processor (e.g., a central processing unit (CPU), a graphics processing unit (GPU), an accelerated processing unit (APU), and/or the like), a microprocessor, a digital signal processor (DSP), and/or any processing component (e.g., a field-programmable gate array (FPGA), an application-specific integrated circuit (ASIC), and/or the like) that can be programmed to perform a function. Memory 606 may include random access memory (RAM), read-only memory (ROM), and/or another type of dynamic or static storage memory (e.g., flash memory, magnetic memory, optical memory, and/or the like) that stores information and/or instructions for use by processor 604.

Storage component 608 may store information and/or software related to the operation and use of computing device 600. For example, storage component 608 may include a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, and/or another type of computer-readable medium, along with a corresponding drive.

Input component 610 may include a component that permits computing device 600 to receive information, such as via user input (e.g., a touch screen display, a keyboard, a keypad, a mouse, a button, a switch, a microphone, and/or the like). Additionally or alternatively, input component 610 may include a sensor for sensing information (e.g., a global positioning system (GPS) component, an accelerometer, a gyroscope, an actuator, and/or the like). Output component 612 may include a component that provides output information from a computing device (e.g., a display, a speaker, one or more light-emitting diodes (LEDs), and/or the like).

Communication interface 614 may include a transceiver-like component (e.g., a transceiver, a separate receiver, and transmitter, etc.) that enables device to communicate with other devices, such as via a wired connection, a wireless connection, or a combination of wired and wireless connections. Communication interface 614 may permit computing device 600 to transmit and/or receive information from another device. For example, communication interface 614 may include an Ethernet interface, an optical interface, a coaxial interface, an infrared interface, a radio frequency (RF) interface, a universal serial bus (USB) interface, a Wi-Fi® interface, a cellular network interface, BLUETOOTH interface, UWB interface, and/or the like. In non-limiting embodiments, communication interface 614 operates through one or both of near-field communication and RFID. Suitable communication protocols and methods for securing communications between communication interface 614 and a communication interface of another device, such as a computing device (e.g., desktop computer, laptop computer, smartphone, smart watch, PDA, tablet, etc.,) can include encryption, e.g., using a secure socket layer (SSL) (e.g., by using public/private key pairs as are known in the art). Additional security protocols are disclosed in, for example, U.S. Pat. Nos. 9,445,264 and 9,463,325, the contents of which are hereby incorporated by reference in their entirety.

A computing device may perform one or more processes described herein. A computing device may perform these processes based on processor 604 executing software instructions stored by a computer-readable medium, such as memory 606 and/or storage component 608, and/or being instructed by a separate computing device. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 606 and/or storage component 608 from another computer-readable medium or from another device via communication interface 614. When executed, software instructions stored in memory 606 and/or storage component 608 may cause processor 604 to perform one or more processes described herein. Additionally or alternatively, hardwired circuitry may be used in place of or in combination with software instructions to perform one or more processes described herein. Thus, embodiments described herein are not limited to any specific combination of hardware circuitry and software.

With reference once again to FIG. 5, in non-limiting embodiments, an exemplary system 500 may include a drug delivery device 502 including short-range communication device 504 as described previously. The drug delivery device 502, through short-range communication device 504, may thus be in communication with a user's computing device 506, such as a smartphone, having its own associated communication interface and processor, as well as memory, storage component, bus, input component, and/or output component.

In non-limiting embodiments, short-range communication device 504 is in one-way communication with a user's computing device 506, e.g., the short-range communication device 504 can only transmit data to the user device 506, and cannot receive data from the user device 506.

As described above, in non-limiting embodiments, upon being placed near drug delivery device 502, user's computing device 506, by virtue of being enabled for NFC, can deliver power to sensor assembly including short-range communication device 504, which can, based on short-range communication device 504 being in electrical communication with capacitive elements, generate and/or record first, second, third, etc. capacitance data which may be transmitted to user's computing device 506, optionally with one or more identifiers (e.g., a device identifier, drug identifier, lot identifier, and/or an identifier associated with short-range communication device 504). In non-limiting embodiments, capacitance data and/or one or more identifiers are encrypted before being transmitted to user device 506. In addition, other sensors can be provided that are in communication with short-range communication device 504 (or any other included communication device, such as an RFID device) and allow for transmission of, for example, GPS data, gyroscope data, temperature data, humidity data, light exposure data, and the like, which may be relevant to any composition that is to be held within drug delivery device 502.

In non-limiting embodiments, user device 506 and/or healthcare system 510, based on software installed thereon, may compare first, second, etc. capacitance data received from drug delivery device 502 to one or more pre-determined capacitance values. Such pre-determined capacitance values may be stored in memory of user device 506 and/or healthcare system 510, and may include predicted or actual capacitance values for various stages of drug delivery. For example, pre-determined capacitance values may include capacitance measured when the syringe is full of a composition (e.g., plastic, glass, a higher density of metal from a compressed needle shield spring, and/or the composition are adjacent capacitive element(s)) (pre-injection), and/or when the syringe has been actuated (e.g., plastic, glass, the rubber stopper, and or a lower density of metal from an expanded spring) are adjacent capacitive element(s)) (post-injection). Results of the comparison(s), as well as received capacitance data and/or one or more identifiers and/or additional data described herein, may be stored on one or both of user device 506 and/or healthcare system 510, and transmitted therebetween via network 508. In non-limiting embodiments, if any capacitance data falls outside of a predetermined range, a message can be generated by or sent to user device 506. In addition, data may be stored by user device 506 and/or healthcare system 510, such that if a particular sensor assembly (e.g., short-range communication device 504) is believed to have not been used previously, but data is detected and/or stored that is indicative of capacitance in a post-use configuration, an alert may be sent to the user device 506 that the drug delivery device 502 may have been previously used or may have been tampered with.

In non-limiting embodiments, healthcare system 510 may be in communication with one or more additional healthcare systems 510. For example, one healthcare system may be maintained by the device manufacturer, and may be in communication with a healthcare system maintained by a study sponsor, pharmaceutical manufacturer, or the like. In non-limiting embodiments, capacitance data (first capacitance data, second capacitance data, etc. and/or any comparisons between the measured capacitance data and predetermined capacitance levels), together with one or more identifiers (e.g., a device identifier and/or an identifier associated with the short-range communication device 504), may be transmitted from device manufacturer system to study sponsor or pharmaceutical manufacturer system, where one or more databases may store data associating the one or more identifiers (e.g., a device identifier and/or an identifier associated with the short-range communication device 504) with a patient identifier, allowing for association of any collected data with the patient.

Although the present disclosure has been described in detail for the purpose of illustration based on what is currently considered to be the most practical and preferred embodiments, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A drug delivery device comprising:
a syringe barrel comprising a proximal end, a distal end, and a sidewall arranged between the proximal end and the distal end defining an interior configured to hold a composition;
a plunger rod received at least partially within the interior of the syringe, the plunger rod comprising a proximal end, a distal end, and a stopper arranged at the distal end; and
a sensor assembly, comprising:
a capacitive element; and
a short-range communication device in electrical communication with the capacitive element, wherein the short-range communication device is capable of measuring capacitance at the capacitive element;
a displaceable needle shield; and
a spring having a compressed configuration and an expanded configuration, the spring configured to displace the needle shield from a first, proximal position in which the displaceable needle shield does not surround a needle of the syringe to a second, distal position in which the displaceable needle shield at least partially surrounds the needle of the syringe,
wherein the capacitive element is configured to detect a change in capacitance based on a change in state of the spring from the compressed configuration to the expanded configuration.

2. The drug delivery device of claim 1, wherein the sensor assembly does not include a power source.

3. The drug delivery device of claim 2, wherein the short-range communication device is an NFC device.

4. The drug delivery device of claim 3, wherein the sensor assembly is powered by an external NFC-enabled device.

5. The drug delivery device of claim 4, wherein the external NFC-enabled device is a mobile device.

6. The drug delivery device of claim 1, wherein the capacitive element comprises a pair of capacitor electrodes.

7. The drug delivery device of claim 1, wherein the capacitive element is arranged at the distal end of the syringe barrel.

8. The drug delivery device of claim 1, wherein the sensor assembly comprises a label arranged on an exterior of the syringe sidewall.

9. The drug delivery device of claim 1, wherein the short-range communication device is a passive RFID chip.

10. (canceled)

11. The drug delivery device of claim 10, wherein the sensor assembly is arranged on the displaceable needle shield.

12. The drug delivery device of claim 11, wherein the capacitive element is arranged on the displaceable needle shield adjacent the spring.

13. A system, comprising:
a drug delivery device comprising:
a syringe barrel comprising a proximal end, a distal end, and a sidewall arranged between the proximal end the distal end defining an interior configured to hold a composition;
a plunger rod received at least partially within the interior of the syringe, the plunger rod comprising a proximal end, a distal end, and a stopper arranged at the distal end; and
a sensor assembly, comprising:
a capacitive element; and
a short-range communication device in electrical communication with the capacitive element, wherein the short range communication device is capable of measuring capacitance at the capacitive element;
a displaceable needle shield; and
a spring having a compressed configuration and an expanded configuration, the spring configured to displace the needle shield from a first, proximal position in which the displaceable needle shield does not surround a needle of the syringe to a second, distal position in which the displaceable needle shield at least partially surrounds the needle of the syringe,
wherein the capacitive element is configured to detect a change in capacitance based on a change in state of the spring from the compressed configuration to the expanded configuration; and
an NFC-enabled mobile device.

14. A method of monitoring drug delivery status, comprising:
providing a drug delivery device comprising:
a syringe barrel comprising a proximal end, a distal end, and a sidewall arranged between the proximal end the distal end defining an interior configured to hold a composition;
a plunger rod received at least partially within the interior of the syringe, the plunger rod comprising a proximal end, a distal end, and a stopper arranged at the distal end; and
a sensor assembly, comprising:
a capacitive element; and
a short-range communication device in electrical communication with the capacitive element, wherein the short range communication device is capable of measuring capacitance at the capacitive element;
a displaceable needle shield; and
a spring having a compressed configuration and an expanded configuration, the spring configured to displace the needle shield from a first, proximal position in which the displaceable needle shield does not surround a needle of the syringe to a second, distal position in which the displaceable needle shield at least partially surrounds the needle of the syringe,
wherein the capacitive element is configured to detect a change in capacitance based on a change in state of the spring from the compressed configuration to the expanded configuration;
powering, with a mobile device, the sensor assembly to obtain second capacitance data; and
transmitting, with the short-range communication device and to the mobile device, the second capacitance data.

15. The method of claim 14, further comprising:
actuating the drug delivery device;
powering, with a mobile device, the sensor assembly to obtain second capacitance data; and
transmitting, with the short-range communication device and to the mobile device the second capacitance data.

16. The system of claim 13, wherein the sensor assembly does not include a power source.

17. The system of claim 16, wherein the short-range communication device is an NFC device.

18. The system of claim 17, wherein the sensor assembly is powered by an external NFC-enabled device.

19. The system of claim 18, wherein the external NFC-enabled device is a mobile device.

20. The system of claim 13, wherein the capacitive element comprises a pair of capacitor electrodes.

21. The system of claim 13, wherein the capacitive element is arranged at the distal end of the syringe barrel.
